(19) United States
(12) Patent Application Publication
(10) Pub. No.: US 2007/0260222 A1
(43) Pub. Date: Nov. 8, 2007

(54) CATHETER HAVING REINFORCING RINGS AND METHOD OF USE

(76) Inventor: Robert G. Kraus, Attleboro, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/382,165

(22) Filed: May 8, 2006

(51) Int. Cl. A61M 25/00

(52) U.S. Cl. 604/527

(57) ABSTRACT

A connector assembly includes a sleeve having a first end and a second end. Reinforcing rings are embedded in the sleeve at the first end. A connector, which has a barbed end, is fluidly connected to the first end of the catheter. During surgery, the first end of the sleeve can be cut to exact length after surgical placement. The barb connector on a shunt housing and the cut end of the catheter are brought together such that the barb advances into the cut end of the catheter and at least two rings snap over the barb, thereby fluidly connecting the shunt housing and the catheter while forming a solid attachment and seal.

### BACKGROUND OF THE INVENTION

1. Field of the Invention

The present invention relates to a catheter having reinforcing rings. More particularly, the present invention relates to a catheter having reinforcing rings that can be cut to length after surgical placement and securely connected to a barbed connector.

2. Discussion of Related Art

There are many applications in which a catheter is fluidly connected to a connector. In medical applications, a catheter is often simply pushed over a barbed connector. But this connection may fail if, for example, an inadvertent force is applied to pull the catheter away from the connector. Thus some have attempted to solve this problem by using an additional connector element radially about the catheter. However these additional elements increase the outer profile of the connection. Additionally, in many embodiments, the additional element is made of a hard plastic material that cannot be treated with currently available antimicrobial impregnation processes. These elements also prevent the surgeon from cutting the length of the catheter to size after it has been surgically placed at a desired location in the body. Thus, the surgeon must make an educated guess at the length of catheter needed before placing it within the body. If the surgeon guesses wrong at the length, removal of that catheter and reinsertion of another catheter is required.

In some instances, the surgeon will suture a catheter that has been connected to a barbed connector to maintain the connection over time. However, suturing a silicone rubber catheter to a barbed connector results in an inconsistent connection. If the suture is too tight, a cut to the catheter can cause leakage and tensile failure. If the suture is too loose, the catheter can be pulled loose and disconnect. Sutures are also subject to degradation over time and can fail years later after being placed within the body. Also, suturing a right angle connection requires the catheter to be lifted away from its preferred location within the body to provide a region for suturing. This is especially true for current shunt housings for treating hydrocephalus that use a right angle connection to the ventricular catheter. After suturing, the catheter is then replaced or pushed further into the body, such as, for example, brain tissue when a ventricular catheter is connected to a shunt housing.

Thus, there is a need in the art for a catheter that has a low outer profile, which can be treated with an antimicrobial impregnation process, and that can be cut to length after surgical placement and securely connected to a barbed connector, without requiring moving the catheter once it has been placed at the desired location within the body.

### SUMMARY OF THE INVENTION

In accordance with a currently preferred exemplary embodiment, the present invention catheter includes a sleeve having a first end and a second end. Reinforcing rings are embedded in the sleeve at the first end.

In accordance with a currently preferred exemplary embodiment, the present invention connector assembly includes a sleeve having a first end and a second end. Reinforcing rings are embedded in the sleeve at the first end. A connector, which has a barbed end, is fluidly connected to the first end of the catheter.

### BRIEF DESCRIPTION OF THE DRAWINGS

The above and still further objects, features and advantages of the present invention will become apparent upon consideration of the following detailed description of a specific embodiment thereof, especially when taken in conjunction with the accompanying drawings wherein like reference numerals in the various figures are utilized to designate like components, and wherein:

FIG. 1 is a perspective view of a catheter having reinforcing rings used with a shunt housing;

FIG. 2 is a partial side view of a right angle connection;

FIG. 3 is a cross-sectional view taken along line 3-3 of FIG. 2 and looking in the direction of the arrows; and

FIG. 4 is a partial perspective view, with parts broken away, of a catheter having reinforcing rings.

### DETAILED DESCRIPTION OF THE CURRENTLY PREFERRED EXEMPLARY EMBODIMENT

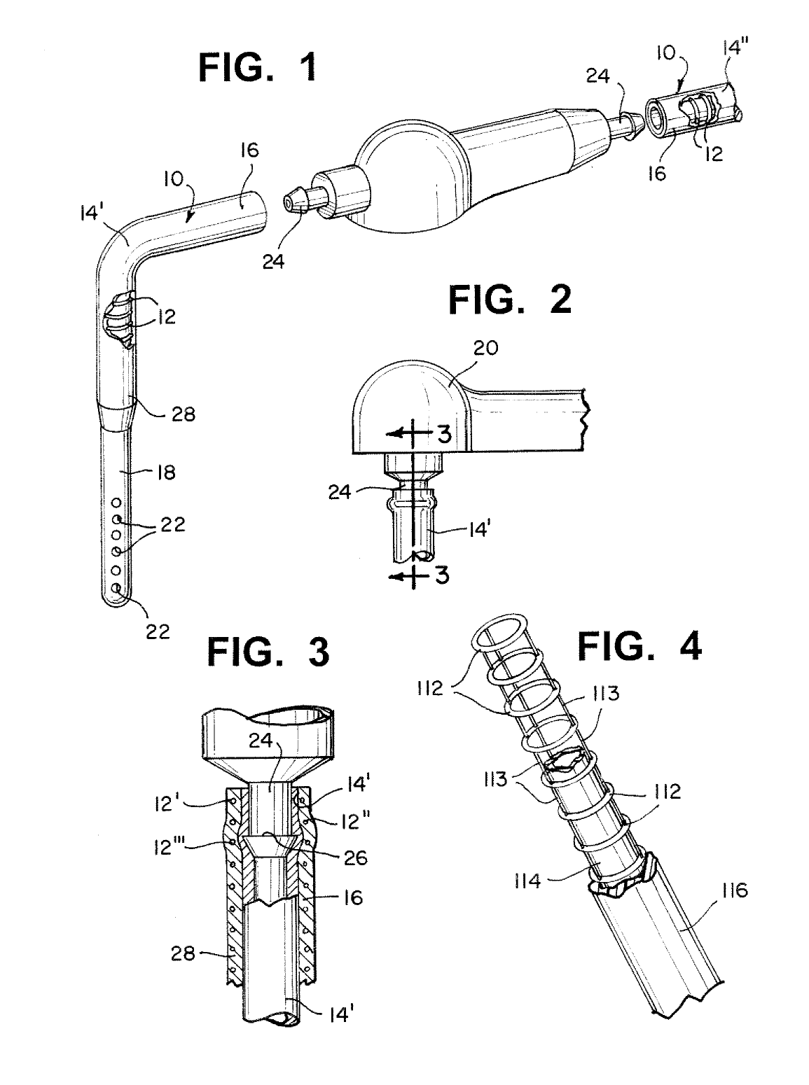

Referring now to FIGS. 1-4, a device and method of using a catheter 10, which has reinforcing rings 12, in accordance with the present invention, is illustrated.

Catheter 10 is in the form of a sleeve 14 and has a first end 16 and a second end 18. A plurality of reinforcing rings 12 are embedded in sleeve 14 at its first end 16. In a currently preferred exemplary embodiment, the catheters are a ventricular catheter 14′ and a drainage catheter 14″ for use with a hydrocephalus shunt valve 20. Ventricular catheter 14′ has at its second end 18 a plurality of inlet openings 22. Inlet openings 22 permit CSF to be drained from the patient's ventricles into the ventricular catheter and delivered to shunt valve 20. Shunt valve 20 regulates the amount of fluid to be drained from the ventricles and then to be delivered to the drainage catheter 14″. Drainage catheter 14′ delivers the cerebrospinal fluid (“CSF”) to a portion of the body that can absorb the CSF. CSF is normally absorbed by the body's venous system. The lower end of the distal catheter of the shunt can be led into the abdomen (ventriculo-peritoneal shunt), wherein CSF passes into the bloodstream.

Reinforcing rings 12 are illustrated as being disposed at regular intervals. However, the rings could be disposed at irregular intervals, such as, for example, becoming closer together as the rings approach the end of the catheter. Another example for spacing the rings at irregular intervals would be to avoid kinking at specific bends in the catheter. In a currently preferred exemplary embodiment there are at least twenty (20) reinforcing rings disposed at regular intervals at the first end of the catheter 10. In a currently preferred embodiment, the ventricular catheter 14′ has about thirty (30) reinforcing rings, and the drainage catheter 14′″ has about ten (10). In some embodiments, the reinforcing rings could be disposed throughout the entire length of the catheter 14. This would be especially practical in embodiments where the length of the catheter is relatively short, such as, for example, less than a two or three inches in length.

Referring now to FIG. 3, at least four (4) reinforcing rings 12 are preferred to effect an adequate connection of the catheter to a barbed connector 24. Preferably, at least two (2) reinforcing rings 12 should pass the barb apex 26 to ensure and optimum attachment. The most proximal ring 12′, near the cut end, may be exposed and may have less adherence to the catheter. Thus, the need to ensure that at least two rings 12 (ie., 12′ and 12″) pass over the barb apex 26. This attachment can be visualized by the surgeon if the catheter sleeve 14 is made of clear silicone. Reinforcing rings 12 preferably have a smaller inside diameter than the largest barb diameter, which is the barb apex.

The catheter sleeve 14 is preferably made of silicone. At least in the area of the reinforcing rings, sleeve 14 is at least partially transparent so that the reinforcing rings 12 can be seen. The reinforcing rings can be made of titanium, stainless steel, or even a rigid plastic material. In fact, just about any material can be used so long as it is MRI compatible and biocompatible. As shown in FIG. 3, the plurality of reinforcing rings 12 are positioned axially discreet from one another. Thus, ventricular catheter 14′ can be relatively easily cut to length after surgical placement and securely connected to a barbed connector. The surgeon's blade can simply cut between adjacent reinforcing rings 12, while still preferably leaving at least four rings to ensure an adequate connection to the barbed connector.

Reinforcing rings 12 are preferably embedded in the sleeve 14 at its first end. Rings 12 can, in one embodiment be embedded in a separate sleeve 28. Sleeve 28 can be placed over the proximal end 16 of each catheter 14 and bonded thereto. Alternatively, the reinforcing rings 12 can be placed on the outer surface of the proximal end 16 of catheter 14 with a slight interference fit. Silicone could then be applied to the outer surface of the proximal end of catheter 14 by, for example, using a dipping process or over molding process. The resulting structure would be a plurality of reinforcing rings embedded in the sleeve at its proximal end.

Referring now to FIG. 4, an alternate embodiment of the reinforcing rings is illustrated. The reinforcing rings 112 can be made as one part, such as, for example, by injection molding. Each ring 112 is connected to an adjacent ring by three attachments 113. The three attachments 113 are frangible by a scalpel. Of course, the three attachments may limit flexibility of the catheter and would be harder to cut than just silicone.

A method of connecting a catheter to a barbed connector, where the catheter is comprised of a sleeve having a first end and a second end, and a plurality of reinforcing rings are embedded in the sleeve at the first end, is described below.

During surgery, the first end can be cut to exact length after ideal surgical placement. The cut end of the catheter is held, possibly with a conforming tool. The barb connector on the shunt housing and the cut end are brought together such that the barb advances into the cut end of the catheter and at least two rings snap over the barb, thereby forming a solid attachment and seal. As the first end of the sleeve is placed over the barbed end of the connector a fluid connection is created between the catheter and the connector. The placing step is sufficient to place at least two reinforcing rings 12′, 12″ passed the apex of the barb. After the placing step, the surgeon can visually verify that at least two reinforcing rings passed the apex of the barb because, at least in the area of the reinforcing rings, the catheter is transparent.

Preferably the method in accordance with the present invention connects a ventricular catheter to a first connector on a shunt housing and connects a drainage catheter to a second connector on the shunt housing. The ventricular catheter is comprised of a sleeve having a first end and a second end, and a plurality of reinforcing rings are embedded in the sleeve at the first end of the ventricular catheter. The drainage catheter is comprised of a sleeve having a first end and a second end, and a plurality of reinforcing rings are embedded in the sleeve at the first end of the drainage catheter. The first and second connector each having a barbed end. The first end of the sleeve of the ventricular catheter is placed over the barbed end of the first connector to fluidly connect the ventricular catheter to the shunt housing. The first end of the sleeve of the drainage catheter is placed over the barbed end of the second connector to fluidly connect the drainage catheter to the shunt housing. The ventricular catheter is placed within the ventricles of the brain. Before the placing of the ventricular catheter, the ventricular catheter is cut to exact size between two reinforcing rings. This cutting step occurs before the step of placing the first end of the sleeve of the ventricular catheter over the barbed end of the first connector.

The catheter with the reinforcing rings embedded therein can be treated with an antimicrobial agent, such as is done with the Bactiseal® catheter that is currently sold by Codman & Shurtleff of Raynham, Mass.

Having described the presently preferred exemplary embodiment of an apparatus and a method of using a catheter having reinforcing rings that can be cut to length after surgical placement and securely connected to a barbed connector, it is believed that other modifications, variations and changes will be suggested to those skilled in the art in view of the teachings set forth herein. Substitutions of elements from one described embodiment to another are also fully intended and contemplated. It is also to be understood that the drawings are not necessarily drawn to scale, but that they are merely conceptual in nature. It is, therefore, to be understood that all such modifications, variations, and changes are believed to fall within the scope of the present invention as defined by the appended claims.

Every issued patent, pending patent application, publication, journal article, book or any other reference cited herein is each incorporated by reference in their entirety.

What is claimed is:

1. A catheter comprising:
a sleeve having a first end and a second end; and
a plurality of reinforcing rings embedded in the sleeve at said first end.

2. The catheter according to claim 1, wherein the plurality of reinforcing rings are disposed at regular intervals.

3. The catheter according to claim 1, wherein there are at least four reinforcing rings.

4. The catheter according to claim 1, wherein there are at least twenty reinforcing rings.

5. The catheter according to claim 1, wherein the sleeve is made of a silicone.

6. The catheter according to claim 5, wherein the silicone, at least in the area of the reinforcing rings, is at least partially transparent.

7. The catheter according to claim 1, wherein the plurality of reinforcing rings are made of titanium.

8. The catheter according to claim 1, wherein the plurality of reinforcing rings are made of stainless steel.

9. The catheter according to claim 1, wherein the plurality of reinforcing rings are made of a rigid plastic material.

10. The catheter according to claim 1, wherein the plurality of reinforcing rings are discreet from one another.

11. The catheter according to claim 2, wherein the plurality of reinforcing rings are discreet from one another.

12. The catheter according to claim 1, wherein the plurality of reinforcing rings are disposed at irregular intervals.

13. The catheter according to claim 1, wherein the plurality of reinforcing rings are interconnected as a one-piece assembly.

14. A connector assembly comprising:
a sleeve having a first end and a second end;
a plurality of reinforcing rings embedded in the sleeve at said first end;
a connector having a barbed end, said first end of said sleeve is connected to said barbed end.

15. The connector assembly according to claim 14, wherein the plurality of reinforcing rings are disposed at regular intervals.

16. The connector assembly according to claim 14, wherein there are at least four reinforcing rings.

17. The connector assembly according to claim 14, wherein there are at least twenty reinforcing rings.

18. The connector assembly according to claim 14, wherein the sleeve is made of a silicone.

19. The connector assembly according to claim 1S, wherein the silicone, at least in the area of the reinforcing rings, is at least partially transparent.

20. The connector assembly according to claim 14, wherein the plurality of reinforcing rings are made of titanium.

21. The connector assembly according to claim 14, wherein the plurality of reinforcing rings are made of stainless steel.

22. The connector assembly according to claim 14, wherein the plurality of reinforcing rings are made of a rigid plastic material.

23. The connector assembly according to claim 14, wherein the plurality of reinforcing rings are discreet from one another.

24. The connector assembly according to claim 15, wherein the plurality of reinforcing rings are discreet from one another.

25. The catheter according to claim 14, wherein the plurality of reinforcing rings are disposed at irregular intervals.

26. The catheter according to claim 14, wherein the plurality of reinforcing rings are interconnected as a one-piece assembly.

27. A method of connecting a catheter to a connector, wherein said catheter is comprised of a sleeve having a first end and a second end, and a plurality of reinforcing rings are embedded in the sleeve at the first end, the connector having a barbed end, said method comprising the step of:
placing the first end of the sleeve over the barbed end of the connector to fluidly connect the catheter to the connector.

28. The method according to claim 27, wherein the placing step is sufficient to place at least two reinforcing rings passed the apex of the barb.

29. The method according to claim 28, wherein said method further comprising the step of after the placing step, verifying that at least two reinforcing rings passed the apex of the barb.

30. The method according to claim 29, wherein the sleeve, at least in the area of the reinforcing rings, is at least partially transparent, said method further comprising the step of, after the placing step, visually verifying that at least two reinforcing rings passed the apex of the barb.

31. A method of connecting a ventricular catheter to a first connector on a shunt housing and connecting a drainage catheter to a second connector on the shunt housing, wherein said ventricular catheter is comprised of a sleeve having a first end and a second end, and a plurality of reinforcing rings are embedded in the sleeve at the first end of the ventricular catheter, said drainage catheter is comprised of a sleeve having a first end and a second end, and a plurality of reinforcing rings are embedded in the sleeve at the first end of the drainage catheter, the first and second connector each having a barbed end, said method comprising the step of:
placing the first end of the sleeve of the ventricular catheter over the barbed end of the first connector to fluidly connect the ventricular catheter to the shunt housing;
placing the first end of the sleeve of the drainage catheter over the barbed end of the second connector to fluidly connect the drainage catheter to the shunt housing.

32. The method according to claim 31, wherein both of the placing steps are sufficient to place at least two reinforcing rings passed the apex of the barb.

33. The method according to claim 32, wherein said method further comprising the step of after the placing steps, verifying that at least two reinforcing rings passed the apex of the barb.

34. The method according to claim 33, wherein the sleeve of both the ventricular catheter and the drainage catheter, at least in the area of the reinforcing rings, is at least partially transparent, said method further comprising the step of, after the pushing steps, visually verifying that at least two reinforcing rings passed the apex of the barb.

35. The method according to claim 31, further comprising the step of placing the ventricular catheter within the ventricals of the brain.

36. The method according to claim 35, further comprising the step of, before the placing the ventricular catheter step, cutting the ventricular catheter between two reinforcing rings.

37. The method according to claim 36, wherein the cutting step occurs before the step of placing the first end of the sleeve of the ventricular catheter over the barbed end of the first connector.

38. The catheter according to claim 1, wherein the catheter has an antimicrobial agent embedded therein.

39. The method according to claim 14, wherein the catheter has an antimicrobial agent embedded therein.
